(19) United States
(12) Patent Application Publication
(10) Pub. No.: US 2026/0112488 A1
(43) Pub. Date: Apr. 23, 2026

(54) INFORMATION PROCESSING APPARATUS AND INFORMATION PROCESSING METHOD

(71) Applicant: NEC Corporation, Tokyo (JP)

(72) Inventor: Kenichiro AKAGI, Tokyo (JP)

(73) Assignee: NEC Corporation, Tokyo (JP)

(21) Appl. No.: 19/352,593

(22) Filed: Oct. 8, 2025

(30) Foreign Application Priority Data
Oct. 23, 2024 (JP) 2024-186946

(51) Int. Cl. G16H 50/20; G16C 20/70

(52) U.S. Cl. CPC G16H 50/20; G16C 20/70

(57) ABSTRACT

An object is to provide a link prediction technology capable of performing highly accurate prediction at lower cost. An information processing apparatus includes an acquisition unit for acquiring a query, a first generation unit for generating one or a plurality of triples with reference to the query, a second generation unit for generating a sentence from each of the one or the plurality of triples, a first calculation unit for acquiring sentence embeddings obtained by using a trained natural language model for each of the generated sentences and calculating a score of each of the sentence embeddings, a second calculation unit for calculating a score of knowledge graph embedding of each of the one or the plurality of triples, and an aggregation unit for aggregating the score calculated by the first calculation unit and the score calculated by the second calculation unit.

### INCORPORATION BY REFERENCE

This application is based upon and claims the benefit of priority from Japanese patent application No. 2024-186946, filed on Oct. 23, 2024, the disclosure of which is incorporated herein in its entirety by reference.

### TECHNICAL FIELD

The present disclosure relates to an information processing apparatus, an information processing method, and a non-transitory computer readable medium.

### BACKGROUND ART

There have been provided various link prediction technologies based on knowledge graphs for predicting genes (proteins) related to diseases such as cancer.

For example, JP 2024-513293 A discloses a technology for achieving link prediction after learning a natural language model from an automatically extracted triple. In addition, JP 2023-522822 A discloses a technology for learning nodes and documents in a knowledge graph.

### SUMMARY

However, in the case of using such a trained model, it is necessary to prepare training data of a sufficient amount of data such as sentences and documents and a natural language model having a high learning cost, and there is a problem that the learning cost increases accordingly.

The present disclosure has been made in view of the above problems, and an example object thereof is to provide a link prediction technology capable of performing highly accurate prediction at lower cost.

An information processing apparatus according to an example aspect of the present disclosure includes an acquisition means for acquiring a query, a first generation means for generating one or a plurality of triples with reference to the query, a second generation means for generating a sentence from each of the one or the plurality of triples, a first calculation means for acquiring sentence embeddings obtained by using a trained natural language model for each of the generated sentences and calculating a score of each of the sentence embeddings, a second calculation means for calculating a score of knowledge graph embedding of each of the one or the plurality of triples, and an aggregation means for aggregating the score calculated by the first calculation means and the score calculated by the second calculation means.

An information processing apparatus according to an example aspect of the present disclosure includes a first generation means for generating a triple group including a positive example triple and a negative example triple with reference to a knowledge graph, a second generation means for generating a sentence from each of the triples included in the triple group, and a first learning means for acquiring sentence embeddings obtained by using a trained natural language model for each of the generated sentences and causing a score calculation model for calculating a score of each of the sentence embeddings to be trained with reference to each of the sentence embeddings.

An information processing method according to an example aspect of the present disclosure includes acquiring a query, generating one or a plurality of triples with reference to the query, generating a sentence from each of the one or plurality of triples, acquiring sentence embeddings obtained by using a trained natural language model for each of the generated sentences and calculating a score of each of the sentence embeddings, calculating a score of knowledge graph embedding of each of the one or plurality of triples, and aggregating a score of the sentence embeddings and a score of the knowledge graph embedding.

An information processing method according to an example aspect of the present disclosure includes: generating a triple group including a positive example triple and a negative example triple with reference to a knowledge graph, generating a sentence from each of the triples included in the triple group, and acquiring sentence embeddings obtained by using a trained natural language model for each of the generated sentences, and causing a score calculation model for calculating a score of each of the sentence embeddings to be trained with reference to each of the sentence embeddings.

A program according to an example aspect of the present disclosure is a program for causing a computer to function as an information processing apparatus, and causes the computer to function as the first generation means, the second generation means, the first calculation means, the second calculation means, and the aggregation means.

A program according to an example aspect of the present disclosure is a program for causing a computer to function as an information processing apparatus, and causes the computer to function as the first generation means, the second generation means, and the first learning means.

According to the present disclosure, as an example of an effect, it is possible to provide a link prediction technique capable of performing prediction with high accuracy at lower cost.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram illustrating a configuration of an information processing apparatus according to the present disclosure;

FIG. 2 is a flowchart illustrating a flow of an information processing method according to the present disclosure;

FIG. 3 is a diagram for describing a basic configuration of a knowledge graph;

FIG. 4 is a block diagram illustrating a configuration of the information processing apparatus according to the present disclosure;

FIG. 5 is a flowchart illustrating a flow of the information processing method according to the present disclosure;

FIG. 6 is a block diagram illustrating a configuration of an information processing system according to the present disclosure;

FIG. 7 is a flowchart illustrating a flow of the information processing method according to the present disclosure;

FIG. 8 is a flowchart illustrating a flow of the information processing method according to the present disclosure;

FIG. 9 is a diagram for describing information processing according to the present disclosure;

FIG. 10 is a diagram for describing information processing according to the present disclosure;

FIG. 11 is a diagram for describing information processing according to the present disclosure;

FIG. 12 is a block diagram illustrating a configuration of the information processing apparatus according to the present disclosure; and

FIG. 13 is a block diagram illustrating a hardware configuration of the information processing apparatus according to the present disclosure.

### EXAMPLE EMBODIMENT

Hereinafter, example embodiments of the present disclosure will be described. However, the present disclosure is not limited to each of the exemplary example embodiments described below, and various modifications can be made within the scope set in the claims. For example, example embodiments obtained by appropriately combining the technologies (some or all of the products or methods) adopted in each of the exemplary example embodiments described below can also fall within the scope of the present disclosure. In addition, example embodiments obtained by appropriately omitting some of the technologies adopted in each of the exemplary example embodiments described below can also fall within the scope of the present disclosure. In addition, the effects mentioned in each of the exemplary example embodiments described below are examples of effects expected in the exemplary example embodiments, and do not define the extension of the present disclosure. That is, example embodiments that do not achieve the effects mentioned in each of the exemplary example embodiments described below can also fall within the scope of the present disclosure.

### First Example Embodiment

A first exemplary example embodiment that is an example embodiment of the present disclosure will be described in detail with reference to the drawings. The present exemplary example embodiment is a basic form of each of the exemplary example embodiments described below. An application range of each technology adopted in the present exemplary example embodiment is not limited to the present exemplary example embodiment. That is, each technology adopted in the present exemplary example embodiment can also be adopted in other exemplary example embodiments included in the present disclosure as long as no particular technical problem occurs. Each technology illustrated in the drawings referred to for describing the present exemplary example embodiment can also be adopted in other exemplary example embodiments included in the present disclosure as long as no particular technical problem occurs.

### Configuration of Information Processing Apparatus 1

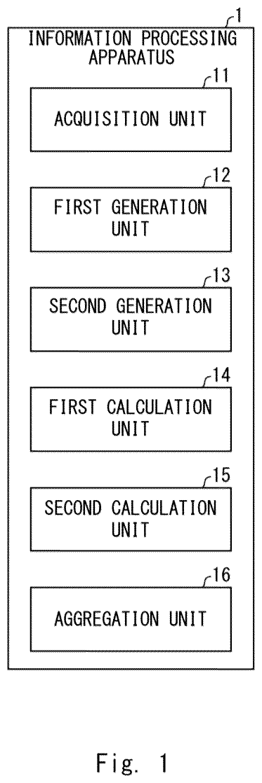

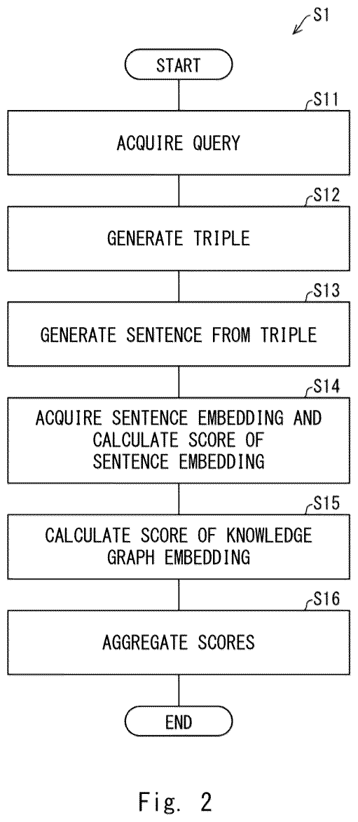

A configuration of an information processing apparatus 1 according to the present exemplary example embodiment will be described with reference to FIG. 1. FIG. 2 is a block diagram illustrating a configuration of the information processing apparatus 1. As illustrated in FIG. 1, the information processing apparatus 1 includes an acquisition unit (means) 11, a first generation unit (means) 12, a second generation unit 13 (means), a first calculation unit (means) 14, a second calculation unit (means) 15, and an aggregation unit (means) 16.

### Acquisition Unit 11

The acquisition unit 11 acquires a query input from a user. Here, as an example, the query may include a plurality of information pieces out of information pieces indicating each of one or a plurality of targets and information pieces indicating a relationship with any of the targets. The acquisition unit 11 extracts these information pieces from the acquired query. As an example, in a case where a query “What protein is highly related to disease 1?” is acquired, the acquisition unit 11 extracts “disease 1” as an information piece indicating a target and “highly related” as an information piece indicating a relationship with the target. Furthermore, the acquisition unit may further extract “protein”as the information piece indicating the target.

The query acquired by the acquisition unit 11 may be expressed as below using the term of the knowledge graph. That is, examples of the query acquired by the acquisition unit 11 include a sentence in which any one of three components of the triple is missing or a sentence in which the abstraction degree of any one of the three components of the triple is relatively high. For example, in the case of the above example, “protein” has a relatively high abstraction degree, and there are various specific examples of protein, and thus the specific example can be an answer to the above query.

Here, the triple is defined by a link serving as a component of the knowledge graph and nodes at both ends of the link. As an example, in a case where the knowledge graph is configured as a directed graph, the triple is configured by (node serving as head, link serving as relation, node serving as tail).

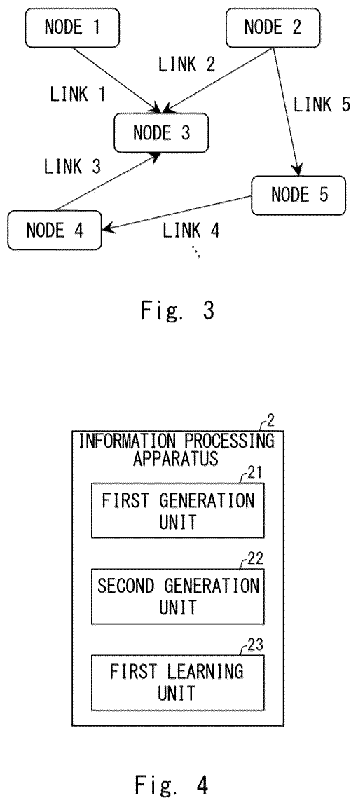

The knowledge graph and the triple will be described below with reference to FIG. 3. FIG. 3 illustrates an example of the knowledge graph. The knowledge graph is a type of data structure, and is constituted by nodes (nodes 1 to 5) and links (links 1 to 5) in the example illustrated in FIG. 3. As an example, the knowledge graph may be expressed as being a data format indicating a connection relationship between people and things. A node may be referred to as a peak, a vertex, an entity, or the like, and the link may be referred to as a relationship, an edge, a relation, or the like.

The minimum record unit constituting the knowledge graph is a set of three (head, relation, tail) (hereinafter, referred to as a triple) representing components and relationships thereof, and the knowledge graph is constructed by listing the triples. Components at both ends (head, tail) correspond to the above-described nodes, and the relationship at the center corresponds to the above-described links. The arrangement order in the triples in FIG. 3 is indicated by a direction of an arrow.

As an example of the query acquired by the acquisition unit 11, as in the above-described example,
in addition to a description such as “What protein is highly related to disease 1?” for performing node prediction,
a description such as “What is the relationship between disease 2 and protein 1?” for performing link prediction may be included.

The format of the query is not particularly limited, and the query may be described in a format applicable to a predetermined format such as describing only the head and tail of the triple or describing only the head and the relation, or the format may be appropriately changed.

### First Generation Unit 12

The first generation unit 12 generates one or a plurality of triples with reference to the query. Here, one or a plurality of triples that can be assumed as a prediction result are generated based on the query input from the user. As an example, in a case where the query is “What protein is highly related to disease 1?”, the first generation unit 12 generates one or a plurality of triples by replacing a missing component or a component having a relatively high abstraction degree, such as (disease 1, highly related, protein 1), (disease 1, highly related, protein 2), . . . , with a specific example (protein 1, protein 2, . . . ). The number of triples generated by the first generation unit 12 for a certain triple may be a predetermined number (e.g., 100) or may be changed according to the acquired triple.

### Second Generation Unit 13

The second generation unit 13 generates a sentence from each of the one or more triples. Here, for example, a sentence such as “Disease 1 and protein 1 are highly related” is generated for the triple of (disease 1, highly related, protein 1), and sentences such as “Prescribe drug 1 for Disease 1” and “Prescribe drug 2 for Disease 1” are generated for the triples of (drug 1, prescribe, disease 1) and (drug 2, prescribe, disease 1), respectively.

### First Calculation Unit 14

The first calculation unit 14 acquires sentence embeddings obtained by using the trained natural language model for each of the generated sentences, and calculates a score of each of the sentence embeddings. Here, “acquiring the sentence embedding” refers to, as an example, acquiring a vector corresponding to the sentence (vectorizing the sentence). Specific examples of the trained natural language model do not limit the present exemplary example embodiment, but include, for example, Sentence-BERT and LLM. As an example, the natural language model may be obtained by acquiring domain knowledge by mainly learning documents in a target field such as medical care and biomedicine. Data of the generated sentence is vectorized by sentence embeddings using such a natural language model, and semantic closeness thereof is quantified. The score of each of the sentence embeddings can be calculated based on the quantified value.

### Second Calculation Unit 15

The second calculation unit 15 calculates a score of knowledge graph embedding of each of the one or a plurality of triples. Knowledge graph embedding is a method of embedding components (triples) of a knowledge graph as vectors, and a score is calculated based on vector values obtained here. In other words, “calculating a score of knowledge graph embedding of a triple” means, as an example, vectorizing the triple and calculating the score of the vector. The processing of knowledge graph embedding can be executed using a trained embedding model as an example.

### Aggregation Unit 16

The aggregation unit 16 aggregates the score calculated by the first calculation unit and the score calculated by the second calculation unit. This aggregation allows the validity of the presence of each triple to be scored (i.e., predicted) with high accuracy. The prediction result by the aggregation unit 16 is, as an example, visually presented to the user via a display unit (not illustrated) or provided to another device via a communication unit (not illustrated). As the score aggregation, for example, an average of two scores may be taken or a weighted average may be taken.

### Effects of Information Processing Apparatus 1

As described above, the information processing apparatus 1 adopts a configuration of:
acquiring a query,
generating one or a plurality of triples with reference to the query,
generating a sentence from each of the one or the plurality of triples,
acquiring sentence embeddings obtained by using a trained natural language model for each of the generated sentences, and calculating a score of each of the sentence embeddings,
calculating a score of knowledge graph embedding of each of the one or the plurality of triples, and
aggregating the score calculated by the first calculation means and the score calculated by the second calculation means.

As described above, in the information processing apparatus 1, a configuration of:
generating a sentence from each of the one or the plurality of triples, and
acquiring sentence embeddings obtained by using a trained natural language model for each of the generated sentences, and calculating a score of each sentence embedding, is adopted, so that increase in cost (temporal cost and cost) can be suppressed. As an example, an increase in cost can be suppressed as compared with a configuration in which a large number of sentences are generated using a trained natural language model.

Furthermore, in the information processing apparatus 1, a configuration of:
aggregating the score calculated by the first calculation means and the score calculated by the second calculation means is adopted, so that highly accurate prediction can be performed by using two pieces of vector information of sentence embeddings and knowledge graph embedding together. Therefore, according to the configuration, a link (or node) prediction technology capable of performing a highly accurate prediction at lower cost is provided.

### Flow of Information Processing Method S1

Next, a flow of an information processing method S1 according to the present exemplary example embodiment will be described with reference to FIG. 2. FIG. 2 is a flowchart illustrating the flow of the information processing method S1. As illustrated in FIG. 2, the information processing method S1 includes a step (processing) S11 of acquiring a query, a step (processing) S12 of generating a triple, a step (processing) S13 of generating a sentence from the triple, a step (processing) S14 of acquiring sentence embeddings and calculating a score of the sentence embedding, a step (processing) S15 of calculating a score of knowledge graph embedding, and a step (processing) S16 of aggregating the scores.

### Step S11

In step S11, the acquisition unit 11 acquires the query. Since a more specific description of the acquisition unit 11 has been described above, the description thereof will be omitted here.

### Step S12

In step S12, the first generation unit 12 generates one or a plurality of triples with reference to the query. Since a more specific description of the first generation unit 12 has been described above, the description thereof will be omitted here.

### Step S13

Subsequently, in step S13, the second generation unit 13 generates a sentence from each of the one or a plurality of triples. Since a more specific description of the second generation unit 13 has been described above, the description thereof will be omitted here.

### Step S14

Subsequently, in step S14, the first calculation unit 14 acquires sentence embeddings obtained by using the trained natural language model for each of the generated sentences, and calculates a score of each of the sentence embeddings. Since a more specific description of the first calculation unit 14 has been described above, the description thereof will be omitted here.

### Step S15

Subsequently, in step S15, the second calculation unit 15 calculates a score of knowledge graph embedding of each of the one or the plurality of triples. Since a more specific description of the second calculation unit 15 has been described above, the description thereof will be omitted here.

### Step S16

Subsequently, in step S16, the aggregation unit 16 aggregates the score calculated by the first calculation unit 14 and the score calculated by the second calculation unit 15. Since a more specific description of the aggregation unit 16 has been described above, the description thereof will be omitted here.

### Effects of Information Processing Method S1

As described above, the information processing method S1 adopts a configuration of:
acquiring a query,
generating one or a plurality of triples with reference to the query,
generating a sentence from each of the one or the plurality of triples,
acquiring sentence embeddings obtained by using a trained natural language model for each of the generated sentences, and calculating a score of each of the sentence embeddings,
calculating a score of knowledge graph embedding of each of the one or the plurality of triples, and
aggregating the score calculated by the first calculation means and the score calculated by the second calculation means. With the above configuration, effects similar to those of the information processing apparatus 1 are obtained.

### Configuration of Information Processing Apparatus 2

Next, a configuration of the information processing apparatus 2 according to the present exemplary example embodiment will be described with reference to FIG. 4. FIG. 4 is a block diagram illustrating the configuration of the information processing apparatus 2. As illustrated in FIG. 4, the information processing apparatus 2 includes a first generation unit 21, a second generation unit 22, and a first learning unit 23.

### First Generation Unit 21

The first generation unit 21 generates a triple group including a positive example triple and a negative example triple with reference to the knowledge graph. Here, one or a plurality of positive example triples are acquired with reference to the knowledge graph. Based on this acquired positive example triple, the first generation unit 21 acquires a predetermined number of negative example triples and generates a predetermined number of triple groups including the one or the plurality of positive example triples and the one or the plurality of negative example triples. The first generation unit 21 may give a label indicating positive example for the positive example triple, and may give a label indicating negative example for the negative example triple. These labels are referred to in learning of a score calculation model SCM to be described later.

### Second Generation Unit 22

The second generation unit 22 generates a sentence from each of the triples included in the triple group. Here, for example, for the triple of (protein 2, interaction, protein 1) and (protein 2, related, disease 1), sentences of “Protein 2 interacts with Protein 1” and “Protein 2 is related to disease 1” are generated.

### First Learning Unit 23

The first learning unit 23 acquires sentence embeddings obtained by using a trained natural language model for each of the generated sentences, and causes a score calculation model for calculating a score of each of the sentence embeddings to be trained with reference to each of the sentence embeddings. As an example, the first learning unit 23:
acquires a vector (sentence embedding) corresponding to each of the generated sentences, and
trains the score calculation model with reference to the vector (sentence embedding) and a label (positive example or negative example) given to a sentence serving as a source of the vector.

As an example, the first learning unit 23 causes the score calculation model to be trained in such a way that the score for the positive example becomes higher than the score for the negative example. Specific examples of the trained natural language model do not limit the present exemplary example embodiment, but include, for example, Sentence-BERT and LLM.

As an example, the natural language model may be obtained by acquiring domain knowledge by mainly learning documents in a target field. Data of the generated sentence is vectorized by sentence embeddings using such a natural language model, and semantic closeness thereof is quantified. Based on the quantified value, the score calculation model can learn with reference to each of the sentence embeddings.

### Effects of Information Processing Apparatus 2

As described above, the information processing apparatus 2 adopts a configuration of:
generating a triple group includes positive example triples and negative example triples with reference to the knowledge graph,
generating a sentence from each of the triples included in the triple group, and
acquiring sentence embeddings obtained by using a trained natural language model for each of the generated sentences, and causing the score calculation model for calculating a score of each of the sentence embeddings to be trained with reference to each of the sentence embeddings.

As described above, in the information processing apparatus 2, a configuration of:
generating a sentence from each of the one or the plurality of triples, and
acquiring sentence embeddings obtained by using a trained natural language model for each of the generated sentences, and calculating a score of each sentence embedding, is adopted, so that increase in cost (temporal cost and cost) can be suppressed. As an example, an increase in cost can be suppressed as compared with a configuration in which a large number of sentences are generated using a trained natural language model.

Furthermore, in the information processing apparatus 2, a configuration of:
aggregating the score calculated by the first calculation means and the score calculated by the second calculation means is adopted, so that highly accurate prediction can be performed by using two pieces of vector information of sentence embeddings and knowledge graph embedding together. Therefore, according to the configuration, a link (or node) prediction technology capable of performing a highly accurate prediction at lower cost is provided.

### Flow of Information Processing Method S2

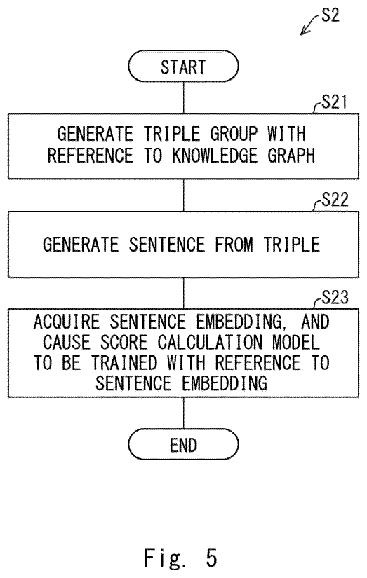

Next, a flow of an information processing method S2 according to the present exemplary example embodiment will be described with reference to FIG. 5. FIG. 5 is a flowchart illustrating the flow of the information processing method S2. As illustrated in FIG. 5, the information processing method S2 includes a step (processing) S21 of generating a triple group with reference to a knowledge graph, a step (processing) S22 of generating a sentence from the triple, and a step (processing) S23 of acquiring sentence embeddings and causing a score calculation model to be trained with reference to the sentence embedding.

### Step S21

In step S21, the first generation unit 21 generates a triple group including a positive example triple and a negative example triple with reference to the knowledge graph. Since a more specific description of the first generation unit 21 has been described above, the description thereof will be omitted here.

### Step S22

Subsequently, in step S22, the second generation unit 22 generates a sentence from each of the triples included in the triple group. Since a more specific description of the second generation unit 22 has been described above, the description thereof will be omitted here.

### Step S23

Subsequently, in step S23, the first learning unit 23 acquires the sentence embeddings obtained by using the trained natural language model for each of the generated sentences, and causes the score calculation model for calculating the score of each of the sentence embeddings to be trained with reference to each of the sentence embeddings. Since a more specific description of the first learning unit 23 has been described above, the description thereof will be omitted here.

### Effects of Information Processing Method S2

As described above, the information processing method S2 adopts a configuration of:
generating a triple group includes positive example triples and negative example triples with reference to the knowledge graph,
generating a sentence from each of the triples included in the triple group, and
acquiring sentence embeddings obtained by using a trained natural language model for each of the generated sentences, and causing the score calculation model for calculating a score of each of the sentence embeddings to be trained with reference to each of the sentence embeddings. With the above configuration, effects similar to those of the information processing apparatus 2 are obtained.

### Second Example Embodiment

A second exemplary example embodiment that is an example embodiment of the present disclosure will be described in detail with reference to the drawings. Components having the same functions as the components described in the above-described exemplary example embodiments are denoted by the same reference numerals, and the description thereof will be appropriately omitted. An application range of each technology adopted in the present exemplary example embodiment is not limited to the present exemplary example embodiment. That is, each technology adopted in the present exemplary example embodiment can also be adopted in other exemplary example embodiments included in the present disclosure as long as no particular technical problem occurs. Each technology illustrated in each of the drawings referred to for describing the present exemplary example embodiment can also be adopted in another exemplary example embodiment included in the present disclosure within a range in which no particular technical problem occurs.

### Configuration of Information Processing System 1A

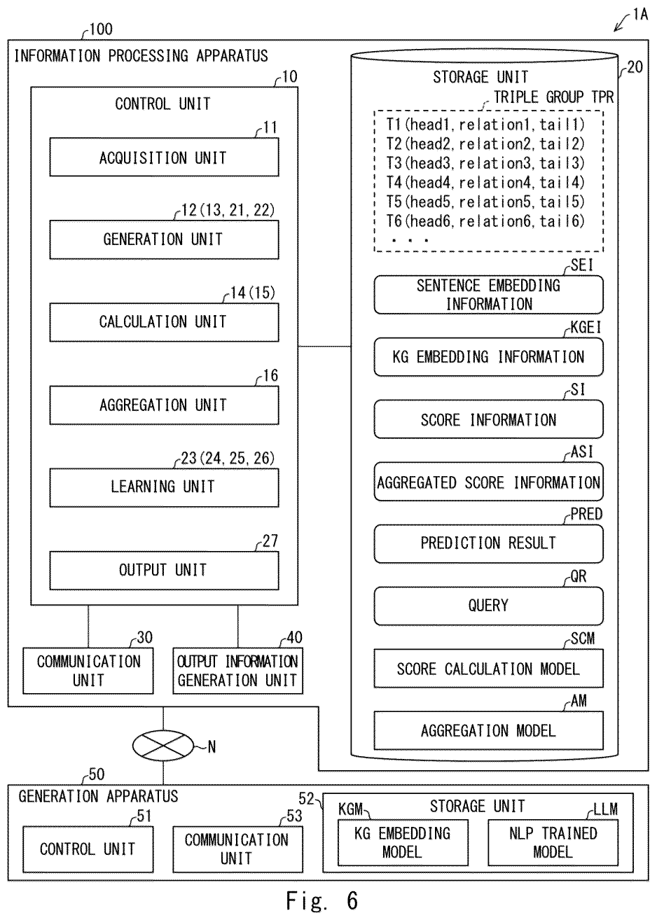

A configuration of an information processing system 1A according to the present exemplary example embodiment will be described with reference to FIG. 6. FIG. 6 is a block diagram illustrating a configuration of an information processing system 1A. As illustrated in FIG. 6, the information processing system 1A includes an information processing apparatus 100 and a generation apparatus 50 connected to the information processing apparatus 100 via a network N. Here, the specific configuration of the network N is not limited to the present exemplary example embodiment, but as an example, a wireless Local Area Network (LAN), a wired LAN, a Wide Area Network (WAN), a public line network, a mobile data communication network, or a combination of these networks can be used.

### Generation Apparatus 50

As illustrated in FIG. 6, the generation apparatus 50 includes a control unit 51, a storage unit 52, and a communication unit 53. The communication unit 53 communicates with an apparatus outside the generation apparatus 50. As an example, the communication unit 53 communicates with the information processing apparatus 100 included in the information processing system 1A. The communication unit 53 transmits data supplied from the control unit 51 to the information processing apparatus 100, and supplies data received from the information processing apparatus 100 to the control unit 51. The data received by the communication unit 53 from the information processing apparatus 100 may include a triple group including positive examples and negative examples generated by the information processing apparatus 100. Furthermore, the data provided by the communication unit 53 to the information processing apparatus 100 can include at least any of link data and node data generated by a natural language trained model to be described later based on the triple group.

The storage unit 52 stores a Knowledge Graph (KG) trained model KGM and a Natural Language Processing (NLP) trained model LLM. As an example, the storage unit 52 stores a plurality of parameters defining these models. These parameters are, as an example, parameters learned in advance through machine learning (parameters subjected to update processing through machine learning), but this does not limit the present exemplary example embodiment. In addition, the natural language trained model is a model trained using a sentence generated from a triple.

The control unit 51 acquires an output result by each model by using the above two models. As an example, the control unit 51 inputs a query received from the information processing apparatus 100 to two models, and acquires embedding data generated by the two models. Furthermore, the data and the score are provided to the information processing apparatus 100 via the communication unit 53.

In the present exemplary example embodiment, the generation apparatus 50 is illustrated as an apparatus separate from the information processing apparatus 100, but this does not limit the present exemplary example embodiment. For example, the control unit of the information processing apparatus 100 may have a function as the control unit 51 included in the generation apparatus 50 or the natural language model execution unit in the control unit 51. Similarly, two or more models stored in the storage unit 52 included in the generation apparatus 50 may be stored in the storage unit of the information processing apparatus 100, and the two or more models may be executable by the information processing apparatus 100 itself.

### Configuration of Information Processing Apparatus 100

Subsequently, a configuration of the information processing apparatus 100 according to the present exemplary example embodiment will be described with reference to FIG. 6. As illustrated in FIG. 6, the information processing apparatus 100 includes a control unit 10, a storage unit 20, a communication unit 30, and an input/output unit 40. The information processing apparatus 100 has a function as a learning apparatus that causes a score calculation model SCM, an aggregation model AM, and the like to be described later to be trained, and a function as an inference apparatus that executes inference processing using the trained score calculation model SCM, aggregation model AM, and the like. The learning processing executed by the information processing apparatus 100 is executed in the learning phase, and the inference processing executed by the information processing apparatus 100 is executed in the inference phase.

### Communication Unit 30

The communication unit 30 communicates with an apparatus outside the information processing apparatus 100. As an example, the communication unit 30 communicates with the generation apparatus 50. The communication unit 30 transmits data supplied from the control unit 10 to the generation apparatus 50, and supplies data received from the generation apparatus 50 to the control unit 10. The data transmitted from the communication unit 30 to the generation apparatus 50 may include the triple acquired by the control unit 10. Furthermore, the data received by the communication unit 30 from the generation apparatus 50 can include the embedding data, the score data, and the like generated by the generation apparatus 50 based on the triple.

### Input/output Unit (Output Information Generation Unit) 40

The input/output unit 40 includes at least one of input/output devices such as a keyboard, a mouse, a display, a printer, and a touch panel. Alternatively, input/output devices such as a keyboard, a mouse, a display, a printer, and a touch panel may be connected to the input/output unit 40. With such a configuration, the input/output unit 40 receives inputs of various types of information to the information processing apparatus 100 from the connected input device. In addition, the input/output unit 40 outputs various types of information to the connected output device under the control of the control unit 10. Examples of the input/output unit 40 include an interface such as, for example, a Universal Serial Bus (USB). The input/output unit 40 may be referred to as an output information generation unit.

### Storage Unit 20

The storage unit 20 stores various types of data to be referred to by the control unit 10 and various types of data generated by the control unit 10. As an example, in the storage unit 20,
Triple group TPR
sentence embeddings information SEI
Knowledge Graph (KG) Embedding Information KGEI
Score Information SI
Aggregated Score Information ASI
Prediction result PRED
Query QR
Score Calculation Model SCM
Aggregation Model AM
   and the like are stored.

Here, the triple group TPR may include one or a plurality of triples generated by the generation unit 12. The sentence embeddings information SEI includes the result of the sentence embeddings (each component of the sentence embeddings vector). The knowledge graph embedding information KGEI includes a result of the knowledge graph embedding (each component of the knowledge graph embedding vector).

The score information includes the score of sentence embeddings and the score of knowledge graph embedding calculated by the calculation unit 14. The aggregated score information ASI includes scores aggregated by the aggregation unit 16. The query QR includes an inquiry (question, instruction) acquired by the acquisition unit 11 from the user. The prediction result PERD is information generated with reference to the aggregated score. The score calculation model SCM is a model for calculating the score of each of the sentence embeddings, and includes one or a plurality of parameters to be learned (to be updated). Furthermore, the aggregation model AM is a model for calculating an aggregated score, and may include, as an example, one or a plurality of parameters to be learned (to be updated). Details of data, information, models, and the like stored in the storage unit 20 will be described later.

### Control Unit 10

As illustrated in FIG. 6, the control unit 10 includes a generation unit 12, a calculation unit 14, an aggregation unit 16, a learning unit 23, and an output unit 27. Here, since the generation unit 12 also has the same functions as the first generation unit 12 and the second generation unit 13 included in the information processing apparatus 1 described in the first exemplary example embodiment and the first generation unit 21 and the second generation unit 22 included in the information processing apparatus 2 described in the first exemplary example embodiment, the generation unit 12 may be referred to as a generation unit 12 (13, 21, 22). Similarly, the calculation unit 14 and the learning unit 23 may also be referred to as a calculation unit 14 (15) and a learning unit 23 (24, 25, 26).

### Acquisition Unit 11

The acquisition unit 11 acquires a query QR input from the user in the inference phase. The acquisition of the query QR may be performed, for example, via the communication unit 30 or via the input/output unit 40. Here, as in the exemplary example embodiment, the query QR may include, as an example, a plurality of information pieces out of information pieces indicating each of one or a plurality of targets and information pieces indicating a relationship with any of the targets. The acquisition unit 11 extracts these information pieces from the acquired query. As an example, in a case where a query “What protein is highly related to disease 1?” is acquired, the acquisition unit 11 extracts “disease 1” as an information piece indicating a target and “highly related” as an information piece indicating a relationship with the target. Furthermore, the acquisition unit may further extract “protein” as the information piece indicating the target.

The query QR acquired by the acquisition unit 11 may be expressed as below using the term of the knowledge graph. That is, examples of the query acquired by the acquisition unit 11 include a sentence in which any one of three components of the triple is missing or a sentence in which the abstraction degree of any one of the three components of the triple is relatively high. For example, in the case of the above example, “protein” has a relatively high abstraction degree, and there are various specific examples of protein, and thus the specific example can be an answer to the above query QR.

As an example of the query QR acquired by the acquisition unit 11, as in the above-described example,
in addition to a description such as “What protein is highly related to disease 1?” for performing node prediction,
a description such as “What is the relationship between disease 2 and protein 148 ” for performing link prediction may be included.

The format of the query QR is not particularly limited, and the query may be described in a format applicable to a predetermined format such as describing only the head and tail of the triple or describing only the head and the relation, or the format may be appropriately changed. The query includes information regarding at least any of a regimen, a chemical agent, a gene, and a disease.

### Generation Unit 12

The first generation unit 21 generates a triple group TPR including a positive example triple and a negative example triple with reference to the knowledge graph in the learning phase. Here, one or a plurality of positive example triples are acquired with reference to the knowledge graph. Based on the acquired positive example triples, the generation unit 12 acquires a predetermined number of negative example triples in the learning phase, and generates a predetermined number of triple groups TPR including one or more positive example triples and one or more negative example triples. The generation unit 12 may give a label indicating positive example for the positive example triple, and may give a label indicating negative example for the negative example triple. These labels are referred to in learning of a score calculation model to be described later.

Here, each triple of the triple group TPR is defined by a link serving as a component of the knowledge graph and nodes at both ends of the link as in the first exemplary example embodiment. As an example, in a case where the knowledge graph is configured as a directed graph, the triple is configured by (node serving as head, link serving as relation, node serving as tail). The knowledge graph and the triple are as described with reference to FIG. 3 in the first exemplary example embodiment.

The minimum record unit constituting the knowledge graph is a set of three (head, relation, tail) (hereinafter, referred to as a triple) representing components and relationships thereof, and the knowledge graph is constructed by listing the triples. Components at both ends (head, tail) correspond to the above-described nodes, and the relationship at the center corresponds to the above-described links. The arrangement order in the triples in FIG. 3 is indicated by a direction of an arrow.

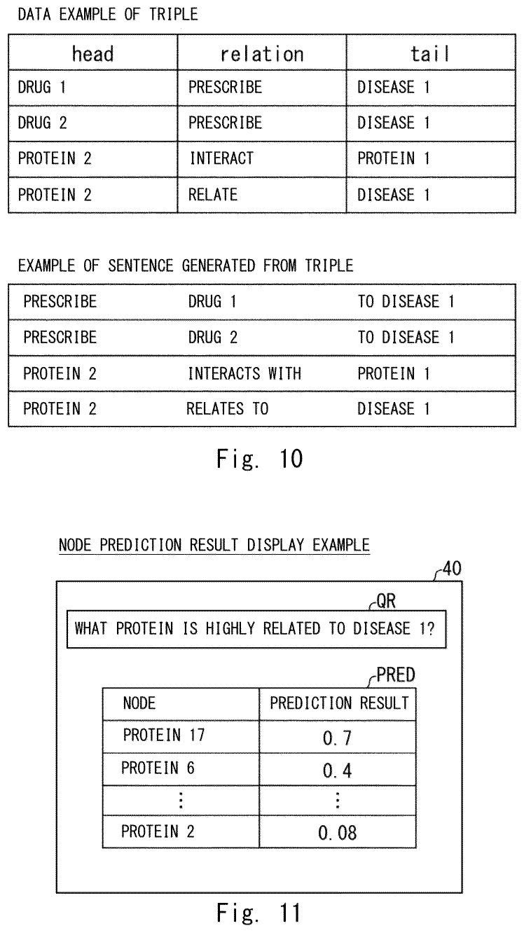

An example of the triple group TPR generated by the generation unit 12 in the learning phase in the present exemplary example embodiment is illustrated in the upper part of FIG. 10. Here, “drug 1”, “drug 2”, and “protein 2” correspond to the head, “prescribe”, “interact”, and “relate”correspond to the relation, and “disease 1”and “protein 1”correspond to the tail.

In addition, in the inference phase, the generation unit 12 generates one or a plurality of triples with reference to the query QR acquired by the acquisition unit 11. Here, one or a plurality of triples generated by the generation unit 12 in the inference phase may also be referred to as a triple group TPR. However, the triple group TPR generated in the inference phase and the triple group TPR generated in the learning phase may generally be different. The triple group TPR generated in the inference phase may be referred to as a triple group TPRI, and the triple group TPR generated in the learning phase may be referred to as a TPRL.

The second generation unit 22 generates a sentence from each of the triples included in the triple group TPR. As an example, the second generation unit 22 generates one or a plurality of sentences with reference to the triple group TPRI generated by the generation unit 12 in the inference phase, and generates one or a plurality of sentences with reference to the triple group TPRL generated by the generation unit 12 in the learning phase. Here, for example, as illustrated in the lower two rows of the lower part of FIG. 10, sentences of “Protein 2 interacts with protein 1” and “Protein 2 is related to disease 1” are generated for the triples of (protein 2, interact, protein 1) and (protein 2, relate, disease 1).

### Calculation Unit 14

In the inference phase, the calculation unit 14 acquires sentence embeddings obtained using a trained natural language model (as an example, the NLP trained model LLM described above) for each of one or a plurality of sentences generated by the generation unit 12, and inputs each of the sentence embeddings to the machine learned score calculation model SCM to calculate a score of the sentence embedding. Here, as in the first exemplary example embodiment, the “sentence embedding” refers to, as an example, a vector corresponding to the sentence (i.e., vectorization of the sentence). Here, it is possible to calculate the score of the sentence embeddings by inputting each of the sentence embeddings to a score calculation model SCM trained by the first learning unit 23 to be described later.

The calculation unit 14 calculates knowledge graph embedding of each of the one or plurality of triples, and refers to the calculated knowledge graph embedding to execute link prediction regarding each of the one or plurality of triples, thereby calculating a score of the knowledge graph embedding. Knowledge graph embedding is a method of embedding components (triples) of a knowledge graph as vectors, and a score is calculated based on vector values obtained here. In other words, “calculating a score of knowledge graph embedding of a triple” means, as an example, vectorizing the triple and calculating the score of the vector. The processing of knowledge graph embedding can be executed using a trained embedding model (KG embedding model KGM described above) by way of an example.

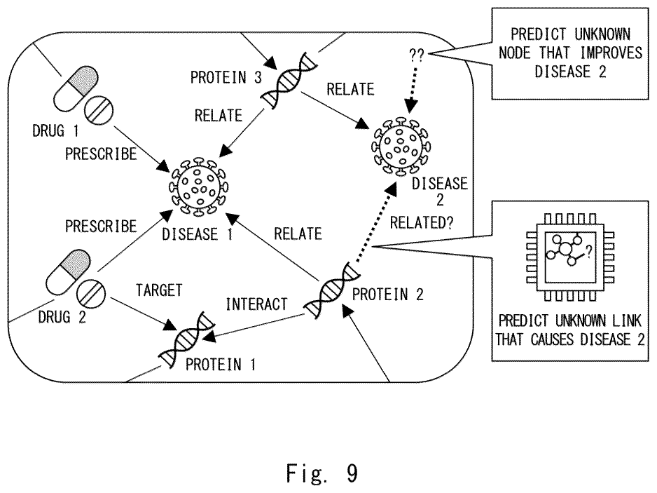

The link prediction is a method of analyzing a knowledge graph, and predicts a link that is not included in data but can be established. For example, in FIG. 9, an unknown link that causes disease 2 is predicted (Is protein 2 related?). Alternatively, not only the link prediction but also a node connected from a certain node by a designated link may be predicted.

Specifically, in FIG. 9, an unknown node that improves disease 2 is predicted.

### Learning Unit 23

In the learning phase, the learning unit 23 acquires the sentence embeddings obtained using the trained natural language model (as an example, the NLP trained model LLM described above) for each of the sentences generated by the generation unit 12, and causes the score calculation model SCM for calculating the score of each of the sentence embeddings to be trained with reference to each of the sentence embeddings. As an example, the learning unit 23:
acquires a vector (sentence embedding) corresponding to each of the generated sentences, and
trains the score calculation model with reference to the vector (sentence embedding) and a label (positive example or negative example) given to a sentence serving as a source of the vector.

As an example, the learning unit 23 causes the score calculation model to be trained in such a way that the score for the positive example becomes higher than the score for the negative example. Specific examples of the trained natural language model do not limit the present exemplary example embodiment, but include, for example, Sentence-BERT and LLM. As an example, the natural language model may be a model obtained by acquiring domain knowledge by mainly learning documents in a target field, or may be a model obtained by machine learning with reference to learning data in a medical field or a biochemical field.

Data of the generated sentence is vectorized by sentence embeddings using such a natural language model, and semantic closeness thereof is quantified. Based on the quantified value, the score calculation model can learn with reference to each of the sentence embeddings. The obtained sentence embeddings are stored in the storage unit 52 as sentence embeddings information SEI. Based on the sentence embeddings information SEI, the score calculation model can learn with reference to each of the sentence embeddings.

Furthermore, in the learning phase, the learning unit 23 causes an embedding model (the KG embedding model KGM described above) for performing knowledge graph embedding to be learned with reference to each of the triples included in the triple group TPR. As described above, knowledge graph embedding is a method of embedding components (triples) of a knowledge graph as vectors, but here, the vectors are learned. The learned vector is stored in the storage unit 52 as KG embedding information KGEI.

As an example, in the learning phase, the learning unit 23 may learn the embedding model KGM with reference to a label (positive example or negative example) given to a sentence that is a source of the vector. Furthermore, as an example, the learning unit 23 causes the embedding model KGM to be trained in such a way that the score for the positive example becomes higher than the score for the negative example.

Furthermore, in the learning phase, the learning unit 23 causes a link prediction model for performing link prediction with reference to the knowledge graph embedding to be learned with reference to each of the triples included in the triple group TPR. Here, the link prediction is a method of analyzing a knowledge graph, and predicts a link that is not included in data but can be established. For example, in FIG. 9, an unknown link that causes disease 2 is predicted (Is protein 2 related?). Alternatively, not only the link prediction but also a node connected from a certain node by a designated link may be predicted. Specifically, in FIG. 9, an unknown node that improves disease 2 is predicted.

The link prediction and the node prediction according to the present exemplary example embodiment are frameworks for predicting a missing link or a missing node in the knowledge graph, and as an example, a plurality of feature amount formats such as latent (learning) feature amount (Latent), relational feature amount (Relational), and numerical feature amount (Numerical) can be integrated and used. These feature amounts can have many real values.

In the link prediction and the node prediction according to the exemplary example embodiment, as an example, combinations of various feature amount formats are possible by integrating the embedding base learning into a stochastic model by two methods. The first method enables the integration of these feature amount formats into an end-to-end differentiable learning system by performing modeling of numerical features with radial basis functions. In the second method, a stochastic expert product (PoE) approach is used to combine feature amount formats. In any relationship, there are separate experts that process each different feature amount format. Then, scores of three sub-models (expert of latent variable, expert of relational feature amount, expert of numerical feature amount) are added and normalized.

The link prediction and the node prediction according to the exemplary example embodiment are knowledge based learning approaches in which combinations of different feature amount formats can be executed and numerical feature amounts, relational feature amounts, and latent feature amounts are combined. It has better accuracy in the set of benchmark data sets, and it is also possible to extract interpretable rule feature amounts. By presenting these rules to the user, the link prediction can be described, and furthermore, the user can perform visual and temporal work. In addition, the present disclosure may be implemented not only in the field of biomedicine such as diseases, proteins, and drugs, but also as general link or node prediction of a correlation between people, a relationship between people and things, a relationship between things and materials, and the like.

In addition, in the learning phase, the learning unit 23 causes the aggregation model AM that calculates the aggregated score from the score calculated by the score calculation model SCM and the score calculated by the link prediction model to be trained. The aggregated score is stored in the storage unit 52 as the aggregated score information ASI. The two scores before aggregation may be stored in the storage unit 52 as the score SI.

In a model that performs a weighted average as an example of an aggregation model, a coefficient of the weighted average may be determined (coefficient may be updated), with reference to at least any of
results of knowledge graph embedding,
results of sentence embedding, and
positive example, negative example label given to the original triple. The aggregation model may be a model that performs simple averaging instead of weighted averaging.

### Aggregation Unit 16

The aggregation unit 16 aggregates the score of the sentence embedding calculated by the calculation unit 14 and the score of the knowledge graph embedding. This aggregation allows the validity of the presence of each triple to be scored (i.e., predicted) with high accuracy. In addition, the aggregation unit 16 generates the prediction result PRED with reference to the aggregated score. The prediction result PRED may include the aggregated score. Furthermore, the prediction result PRED by the aggregation unit 16 is visually presented to the user via a display unit (not illustrated) or provided to another device via a communication unit (not illustrated) as an example.

### Output Unit 27

The output unit 27 presents the prediction result PRED derived by the aggregation unit 16 to the user via the output information generation unit 40. As an example, the output unit 27 may be configured to visually present the prediction result PRED illustrated in FIG. 11 to the user via the output information generation unit 40. For example, the output information generation unit 40 may refer to the score aggregated by the aggregation unit 16 and generate output information for assisting a medical worker in decision making.

### Result Display Example

“Link prediction” corresponds to giving a sentence in which the relation among the three components of the triple is missing as a query, and complementing a blank. In addition, “node prediction” corresponds to acquiring a sentence in which the head or the tail is missing among the three components of the triple as a query and complementing the blank. For example, a node prediction result as illustrated in FIG. 11 is obtained.

In the example illustrated in FIG. 11, a query QR of “What protein is highly related to disease 1?” is acquired by the user and is acquired by the acquisition unit 11. Answers (nodes) to this query QR are displayed as protein 17, protein 6 . . . . These answers correspond to the scores after aggregation by the aggregation unit 16 described above. In other words, in the example illustrated in FIG. 11, the aggregation unit 16 ranks the nodes associated with the scores after the aggregation in descending order of the aggregated scores (in such a way that larger aggregated scores are at higher rank), and generates the prediction result PRED including the result of the ranking. Such ranking display can assist in a target search of an initial phase of a drug discovery process by drug discovery researchers.

### Flow of Information Processing Method S3

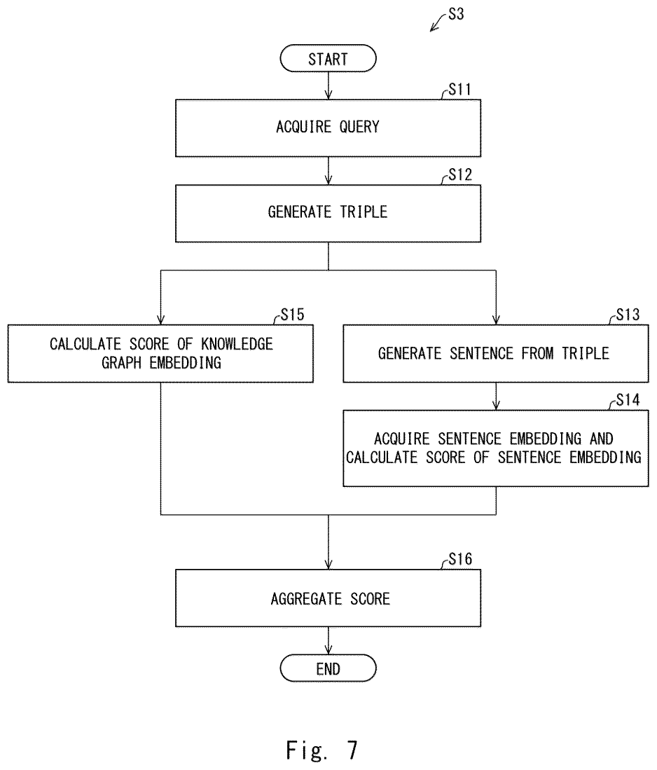

Next, a flow of an information processing method S3 according to the present exemplary example embodiment will be described with reference to FIG. 7. FIG. 7 is a flowchart illustrating the flow of the information processing method S3 executed by the information processing apparatus 100 according to the present exemplary example embodiment, and corresponds to the flow in the inference phase. Each step of the information processing method S3 is similar to each step S11 to S16 of the information processing method S1 according to the first exemplary example embodiment by way of an example, but in the information processing method S3, steps S13 and S14 can be processed in parallel with step S15.

### Step S11

In step S11, the acquisition unit 11 acquires the query QR. Since a more specific description of the acquisition unit 11 has been described above, the description thereof will be omitted here.

### Step S12

In step S12, the generation unit 12 generates one or a plurality of triples (triple group TPRI) with reference to the query QR. Since a more specific description of the generation unit 12 related to this step has been described above, the description thereof will be omitted here.

### Step S13

Subsequently, in step S13, the generation unit 12 generates a sentence from each of the one or a plurality of triples. Since a more specific description of the generation unit 12 related to this step has been described above, the description thereof will be omitted here.

### Step S14

Subsequently, in step S14, the calculation unit 14 acquires sentence embeddings obtained by using the trained natural language model for each of the generated sentences, and calculates a score of each of the sentence embeddings. Since a more specific description of the calculation unit 14 related to this step has been described above, the description thereof will be omitted here.

### Step S15

Subsequently, in step S15, the calculation unit 14 calculates a score of knowledge graph embedding of each of the one or the plurality of triples. Since a more specific description of the calculation unit 14 related to this step has been described above, the description thereof will be omitted here.

### Step S16

Subsequently, in step S16, the aggregation unit 16 aggregates the score of the sentence embedding calculated by the calculation unit 14 and the score of the knowledge graph embedding. Since a more specific description of the aggregation unit 16 has been described above, the description thereof will be omitted here.

As described above, the information processing apparatus 200 includes:
an acquisition means (acquisition unit 11) for acquiring a query,
a first generation means (first generation unit 12 (generation unit 12)) for generating one or a plurality of triples with reference to the query,
a second generation means (second generation unit 13 (generation unit 12)) for generating a sentence from each of the one or plurality of triples,
a first calculation means (first calculation unit 14 (calculation unit 14)) for acquiring sentence embeddings obtained by using a trained natural language model for each of the generated sentences and calculating a score of each of the sentence embeddings,
a second calculation means (second calculation unit 15 (calculation unit 14)) for calculating a score of knowledge graph embedding of each of the one or plurality of triples, and
an aggregation means (aggregation unit 16) for aggregating the score calculated by the first calculation means and the score calculated by the second calculation means. This configuration also achieves various effects according to each of the exemplary example embodiments described above.

According to the above configuration, effects similar to those of the information processing apparatuses 1 and 2 according to the first exemplary example embodiment are obtained. As an example, this can assist the target search of the initial phase of the drug discovery process and the test process of the WET test, and contribute to reduction of the total development cost.

### Flow of Information Processing Method S4

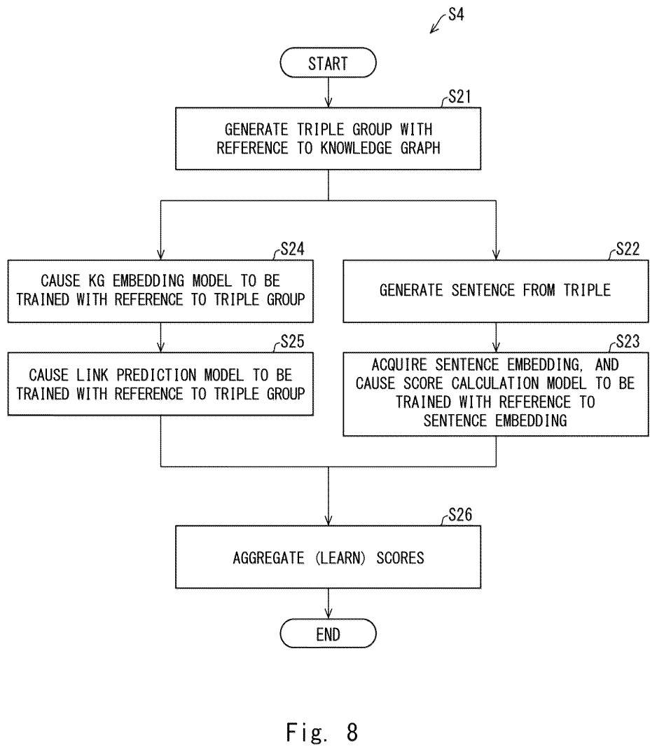

Next, a flow of an information processing method S3 according to the present exemplary example embodiment will be described with reference to FIG. 8. FIG. 8 is a flowchart illustrating the flow of the information processing method S4, and corresponds to the flow in the learning phase. As illustrated in FIG. 8, the information processing method S4 includes, in addition to the above-described steps (processing) S21, S22, and S23, a step (processing) S24 of causing the KG embedding model KGM to be trained with reference to the triple group TPR (as an example, the triple group TPRL), a step (processing) S25 of causing the link prediction model to be trained with reference to the triple group TPR (as an example, the triple group TPRL), and a step (processing) S26 of causing the score aggregation model AM to be trained.

As illustrated in this figure, steps S24 and S25 are processed in parallel with steps S22 and S23, but are not limited thereto, and for example, may be processed in series.

### Step S21

In step S21, the generation unit 12 generates a triple group TPRL including a positive example triple and a negative example triple with reference to the knowledge graph. Since a more specific description of the generation unit 12 related to this step has been described above, the description thereof will be omitted here.

### Step S22

Subsequently, in step S22, the generation unit 12 generates a sentence from each of the triples included in the triple group TPRL. Since a more specific description of the generation unit 12 related to this step has been described above, the description thereof will be omitted here.

### Step S23

Subsequently, in step S23, the learning unit 23 acquires the sentence embeddings obtained by using the trained natural language model for each of the generated sentences, and causes the score calculation model SCM for calculating the score of each of the sentence embeddings to be trained with reference to each of the sentence embeddings. Since a more specific description of the learning unit 23 related to this step has been described above, the description thereof will be omitted here.

### Step S24

In step S24, the learning unit 23 causes the embedding model KGM for performing knowledge graph embedding to be trained with reference to each of the triples included in the triple group TPRL. Since a more specific description of the learning unit 23 related to this step has been described above, the description thereof will be omitted here.

### Step S25

In step S25, the learning unit 23 causes the link prediction model for performing link prediction with reference to the knowledge graph embedding to be trained with reference to each of the triples included in the triple group TPRL. Since a more specific description of the learning unit 23 related to this step has been described above, the description thereof will be omitted here.

### Step S26

Subsequently, in step S26, the learning unit 23 causes the aggregation model AM for calculating the aggregated score from the score calculated by the score calculation model SCM and the score calculated by the link prediction model to be trained.

As described above, the information processing method S4 adopts a configuration of:
generating a triple group including positive example triples and negative example triples with reference to the knowledge graph,
generating a sentence from each of the triples included in the triple group,
acquiring sentence embeddings obtained by using a trained natural language model for each of the generated sentences and causing a score calculation model for calculating a score of each of the sentence embeddings to be trained with reference to each of the sentence embeddings,
causing an embedding model for performing knowledge graph embedding to be trained with reference to each of the triples included in the triple group,
causing a link prediction model for performing link prediction with reference to the knowledge graph embedding to be trained with reference to each of the triples included in the triple group, and
causing an aggregation model for calculating an aggregated score from the score calculated by the score calculation model and the score calculated by the link prediction model to be trained. According to the above configuration, effects similar to those of the information processing apparatuses 1 and 2 according to the first exemplary example embodiment are obtained. As an example, this can assist the target search of the initial phase of the drug discovery process and the test process of the WET test, and contribute to reduction of the total development cost.

### Third Example Embodiment

A third exemplary example embodiment that is an example of the example embodiment of the present disclosure will be described in detail with reference to the drawings. Components having the same functions as the components described in the above-described exemplary example embodiments are denoted by the same reference numerals, and the description thereof will be appropriately omitted. An application range of each technology adopted in the present exemplary example embodiment is not limited to the present exemplary example embodiment. That is, each technology adopted in the present exemplary example embodiment can also be adopted in other exemplary example embodiments included in the present disclosure as long as no particular technical problem occurs. Each technology illustrated in each of the drawings referred to for describing the present exemplary example embodiment can also be adopted in another exemplary example embodiment included in the present disclosure within a range in which no particular technical problem occurs.

### Configuration of Information Processing Apparatus 200

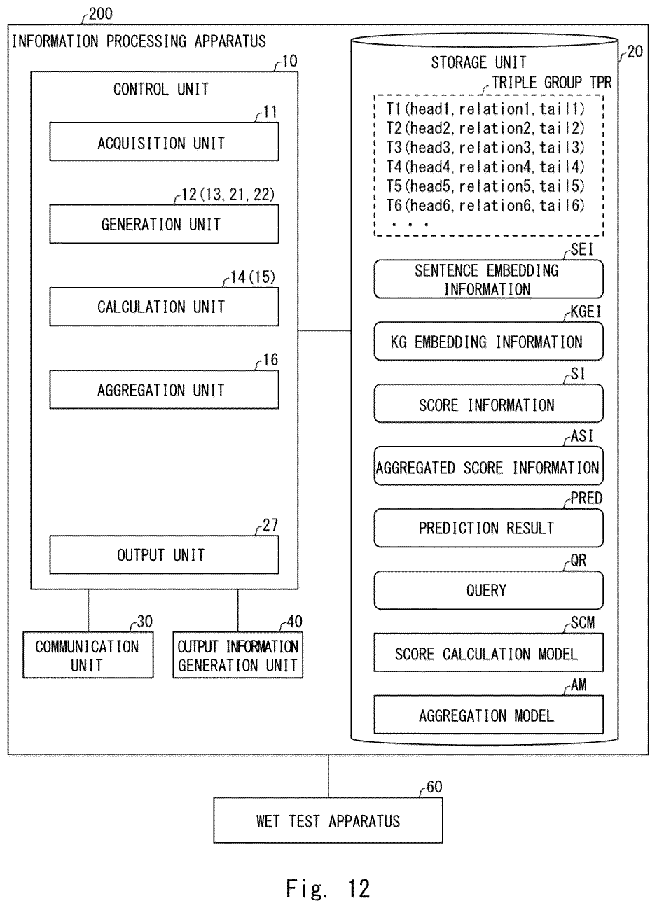

A configuration of an information processing apparatus 200 according to the present exemplary example embodiment will be described with reference to FIG. 12. As illustrated in FIG. 12, the information processing apparatus 200 does not include the learning unit 23 among the configurations included in the information processing apparatus 100 according to the second exemplary example embodiment, but the other configurations are similar to those of the information processing apparatus 100 according to the second exemplary example embodiment. Furthermore, in the present configuration example, the information processing apparatus 200 is connected to a WET test apparatus 60 that performs a WET test via a network N. Here, the term “WET test” refers to a test actually using a protein, a reagent, or the like, and is used as a meaning of comparison with a DRY test such as data analysis. In other words, the WET test apparatus 60 is a device that executes a test actually using a protein, a reagent, or the like.

The output information generation unit (input/output unit) 40 included in the information processing apparatus 200 may refer to the prediction result PRED generated by the aggregation unit 16 and generate instruction information indicating test contents to be executed by the WET test apparatus 60. Then, the output information generation unit 40 transmits the generated instruction information to the WET test apparatus 60 via the network N. Then, the WET test apparatus 60 refers to the instruction information provided from the information processing apparatus 200 and executes the test indicated by the instruction information.

As an example, in a case where the prediction information generated by the output information generation unit 40 is the prediction result PRED illustrated in FIG. 11, the output information generation unit 40 may instruct the WET test apparatus 60 to perform a test using the highest ranking protein 17. Alternatively, the output information generation unit 40 may be configured to give a priority having a positive correlation with the aggregated score (0.7, 0.4, . . . in FIG. 11) to a corresponding node (protein 17, protein 6, . . . ) and instruct the WET test apparatus to execute the test in an order according to the priority. Such instructions may, as an example, assist evaluation tests such as WET tests of genomic researchers.

### Achievement Example by Software

Some or all of the functions of the information processing apparatuses 1, 2, 100, and 200 (hereinafter, also referred to as “each of the above apparatuses”) may be implemented by hardware such as an integrated circuit (IC chip) or may be implemented by software.

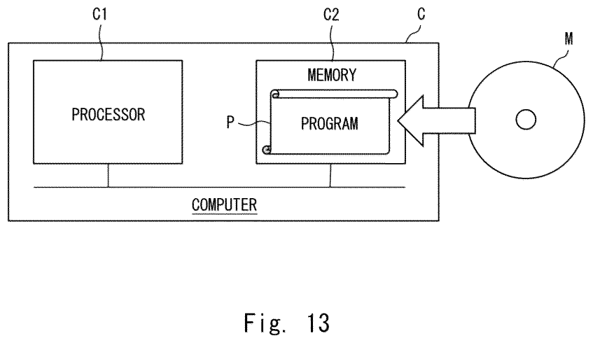

In the latter case, each of the above devices is achieved by, for example, a computer that executes a command of a program as software for achieving each function. An example of such a computer (hereinafter described as a computer C) is illustrated in FIG. 13. FIG. 13 is a block diagram illustrating a hardware configuration of the computer C functioning as each of the above devices.

The computer C includes at least one processor C1 and at least one memory C2. A program P causing the computer C to operate as each of the above devices is recorded in the memory C2. In the computer C, by the processor C1 reading the program P from the memory C2 and executing the program P, each function of each of the above devices is achieved.

As the processor C1, for example, a Central Processing Unit (CPU), a Graphic Processing Unit (GPU), a Digital Signal Processor (DSP), a Micro Processing Unit (MPU), a Floating point number Processing Unit (FPU), a Physics Processing Unit (PPU), a Tensor Processing Unit (TPU), a quantum processor, a microcontroller, or a combination of these can be used. As the memory C2, for example, a flash memory, a Hard Disk Drive (HDD), a Solid State Drive (SSD), or a combination of these can be used.

The computer C may further include a Random Access Memory (RAM) for loading the program P at the time of execution and temporarily storing various types of data. The computer C may further include a communication interface for transmitting and receiving data to and from another device. The computer C may further include an input/output interface for connecting input/output devices such as a keyboard, a mouse, a display, and a printer.

The program P can be recorded in a non-transitory tangible recording medium M readable by the computer C. As such a recording medium M, for example, a tape, a disk, a card, a semiconductor memory, or a programmable logic circuit can be used. The computer C can acquire the program P via such a recording medium M. The program P can be transmitted via a transmission medium. As such a transmission medium, for example, a communication network or a broadcast wave can be used. The computer C can also acquire the program P via such a transmission medium.

### Supplementary Note A

The present disclosure includes the technologies described in the following supplementary notes. However, the present disclosure is not limited to the technologies described in the following supplementary notes, and various modifications can be made within the scope described in the claims.

### Supplementary Note A1

An information processing apparatus including:
an acquisition means for acquiring a query,
a first generation means for generating one or a plurality of triples with reference to the query,
a second generation means for generating a sentence from each of the one or the plurality of triples,
a first calculation means for acquiring sentence embeddings obtained by using a trained natural language model for each of the generated sentences and calculating a score of each of the sentence embeddings,
a second calculation means for calculating a score of knowledge graph embedding of each of the one or the plurality of triples, and
an aggregation means for aggregating the score calculated by the first calculation means and the score calculated by the second calculation means.

### Supplementary Note A2

The information processing apparatus according to supplementary note A1, in which the first calculation means calculates a score of the sentence embeddings by inputting each of the sentence embeddings to a machine learned score calculation model.

### Supplementary Note A3

The information processing apparatus according to supplementary note A2,
in which the second calculation means,
calculates a knowledge graph embedding of each of the one or the plurality of triples, and
calculates the score by executing link prediction related to each of the one or the plurality of triples with reference to the calculated knowledge graph embedding.

### Supplementary Note A4

The information processing apparatus according to any one of supplementary notes A1 to A3, in which the natural language model is a model machine learned with reference to learning data in a medical field or a biochemical field.

### Supplementary Note A5

The information processing apparatus according to any one of supplementary notes A1 to A3, in which the query includes information regarding at least any of a regimen, a chemical agent, a gene, and a disease.

### Supplementary Note A6

The information processing apparatus according to any one of supplementary notes A1 to A3, further including an output information generation means for generating output information for assisting a medical worker in decision making with reference to the score aggregated by the aggregation means.

### Supplementary Note A7

An information processing apparatus including:
a first generation means for generating a triple group including a positive example triple and a negative example triple with reference to a knowledge graph,
a second generation means for generating a sentence from each of the triples included in the triple group, and
a first learning means for acquiring sentence embeddings obtained by using a trained natural language model for each of the generated sentences and causing a score calculation model for calculating a score of each of the sentence embeddings to be trained with reference to each of the sentence embeddings.

### Supplementary Note A8

The information processing apparatus according to supplementary note A7, further including:
a second learning means for causing an embedding model for performing knowledge graph embedding to be trained with reference to each of the triples included in the triple group, and
a third learning means for causing a link prediction model for performing link prediction with reference to the knowledge graph embedding to be trained with reference to each of the triples included in the triple group.

### Supplementary Note A9

The information processing apparatus according to supplementary note A8, further including a fourth learning means for causing an aggregation model for calculating an aggregated score from the score calculated by the score calculation model and the score calculated by the link prediction model to be trained.

### Supplementary Note B

The present disclosure includes the technologies described in the following supplementary notes. However, the present disclosure is not limited to the technologies described in the following supplementary notes, and various modifications can be made within the scope described in the claims.

### Supplementary Note B1

An information processing method including:
an acquisition processing in which at least one processor acquires a query,
a first generation processing in which the at least one processor generates one or a plurality of triples with reference to the query,
a second generation processing in which the at least one processor generates a sentence from each of the one or the plurality of triples,
a first calculation processing in which the at least one processor acquires sentence embeddings obtained by using a trained natural language model for each of the generated sentences and calculates a score of each of the sentence embeddings,
a second calculation processing in which the at least one processor calculates a score of knowledge graph embedding of each of the one or the plurality of triples, and
an aggregation processing in which the at least one processor aggregates the score calculated by the first calculation processing and the score calculated by the second calculation processing.

### Supplementary Note B2

The information processing method according to supplementary note B1, in which the first calculation processing includes calculating a score of the sentence embeddings by inputting each of the sentence embeddings to a machine learned score calculation model.

### Supplementary Note B3

The information processing method according to supplementary note B2, in which the second calculation processing includes:
calculating a knowledge graph embedding of each of the one or the plurality of triples, and
calculating the score by executing link prediction related to each of the one or the plurality of triples with reference to the calculated knowledge graph embedding.

### Supplementary Note B4

The information processing method according to any one of supplementary notes B1 to B3, in which the natural language model is a model machine learned with reference to learning data in a medical field or a biochemical field.

### Supplementary Note B5

The information processing method according to any one of supplementary notes B1 to B3, in which the query includes information regarding at least any of a regimen, a chemical agent, a gene, and a disease.

### Supplementary Note B6

The information processing method according to any one of supplementary notes B1 to B3, further including output information generation processing in which the at least one processor generates output information for assisting a medical worker in decision making with reference to the score aggregated by the aggregation processing.

### Supplementary Note B7

An information processing method including:
a first generation processing in which the at least one processor generates a triple group including a positive example triple and a negative example triple with reference to a knowledge graph,
a second generation processing in which the at least one processor generates a sentence from each of the triples included in the triple group, and
a first learning processing in which the at least one processor acquires sentence embeddings obtained by using a trained natural language model for each of the generated sentences and causes a score calculation model for calculating a score of each of the sentence embeddings to be trained with reference to each of the sentence embeddings.

### Supplementary Note B8

The information processing method according to supplementary note B7, further including:
a second learning processing in which the at least one processor causes an embedding model for performing knowledge graph embedding to be trained with reference to each of the triples included in the triple group, and
a third learning processing in which the at least one processor causes a link prediction model for performing link prediction with reference to the knowledge graph embedding to be trained with reference to each of the triples included in the triple group.

### Supplementary Note B9

The information processing method according to supplementary note B8, further including a fourth learning processing in which the at least one processor causes an aggregation model for calculating an aggregated score from the score calculated by the score calculation model and the score calculated by the link prediction model to be trained.

### Supplementary Note C

The present disclosure includes the technologies described in the following supplementary notes. However, the present disclosure is not limited to the technologies described in the following supplementary notes, and various modifications can be made within the scope described in the claims.

### Supplementary Note C1

An information processing program for causing a computer to function as:
an acquisition means for acquiring a query,
a first generation means for generating one or a plurality of triples with reference to the query,
a second generation means for generating a sentence from each of the one or the plurality of triples,
a first calculation means for acquiring sentence embeddings obtained by using a trained natural language model for each of the generated sentences and calculating a score of each of the sentence embeddings,
a second calculation means for calculating a score of knowledge graph embedding of each of the one or the plurality of triples, and
an aggregation means for aggregating the score calculated by the first calculation means and the score calculated by the second calculation means.

### Supplementary Note C2

The information processing program according to supplementary note C1, in which the first calculation means calculates a score of the sentence embeddings by inputting each of the sentence embeddings to a machine learned score calculation model.

### Supplementary Note C3

The information processing program according to supplementary note C2, in which the second calculation means:
calculates a knowledge graph embedding of each of the one or the plurality of triples, and
calculates the score by executing link prediction related to each of the one or the plurality of triples with reference to the calculated knowledge graph embedding.

### Supplementary Note C4

The information processing program according to any one of supplementary notes C1 to C3, in which the natural language model is a model machine learned with reference to learning data in a medical field or a biochemical field.

### Supplementary Note C5

The information processing program according to any one of supplementary notes C1 to C3, in which the query includes information regarding at least any of a regimen, a chemical agent, a gene, and a disease.

### Supplementary Note C6

An information processing program according to any one of supplementary notes C1 to C3, further causing the computer to function as an output information generation means for generating output information for assisting a medical worker in decision making with reference to the scores aggregated by the aggregation means.

### Supplementary Note C7

An information processing program for causing a computer to function as:
a first generation means for generating a triple group including a positive example triple and a negative example triple with reference to a knowledge graph,
a second generation means for generating a sentence from each of the triples included in the triple group, and
a first learning means for acquiring sentence embeddings obtained by using a trained natural language model for each of the generated sentences and causing a score calculation model for calculating a score of each of the sentence embeddings to be trained with reference to each of the sentence embeddings.

### Supplementary Note C8

The information processing program according to supplementary note C7, further causing the computer to function as:
a second learning means for causing an embedding model for performing knowledge graph embedding to be trained with reference to each of the triples included in the triple group, and
a third learning means for causing a link prediction model for performing link prediction with reference to the knowledge graph embedding to be trained with reference to each of the triples included in the triple group.

### Supplementary Note C9

The information processing program according to supplementary note C8, further causing the computer to function as a fourth learning means for causing an aggregation model for calculating an aggregated score from the score calculated by the score calculation model and the score calculated by the link prediction model to be trained.

### Supplementary Note D

The present disclosure includes the technologies described in the following supplementary notes. However, the present disclosure is not limited to the technologies described in the following supplementary notes, and various modifications can be made within the scope described in the claims.

### Supplementary Note D1

An information processing apparatus that executes:
an acquisition processing of acquiring a query,
a first generation processing of generating one or a plurality of triples with reference to the query,
a second generation processing of generating a sentence from each of the one or the plurality of triples,
a first calculation processing of acquiring sentence embeddings obtained by using a trained natural language model for each of the generated sentences and calculating a score of each of the sentence embeddings,
a second calculation processing of calculating a score of knowledge graph embedding of each of the one or the plurality of triples, and
an aggregation processing of aggregating the score calculated by the first calculation processing and the score calculated by the second calculation processing.

The information processing apparatus may further include a memory. The memory may store a program for causing the at least one processor to execute each of the processing.

### Supplementary Note D2

The information processing apparatus according to supplementary note D1, in which the first calculation processing includes calculating a score of the sentence embeddings by inputting each of the sentence embeddings to a machine learned score calculation model.

### Supplementary Note D3

The information processing apparatus according to supplementary note D2, in which the second calculation processing includes:
calculating a knowledge graph embedding of each of the one or the plurality of triples, and
calculating the score by executing link prediction related to each of the one or the plurality of triples with reference to the calculated knowledge graph embedding.

### Supplementary Note D4

The information processing apparatus according to any one of supplementary notes D1 to D3, in which the natural language model is a model machine learned with reference to learning data in a medical field or a biochemical field.

### Supplementary Note D5

The information processing apparatus according to any one of supplementary notes D1 to D3, in which the query includes information regarding at least any of a regimen, a chemical agent, a gene, and a disease.

### Supplementary Note D6

The information processing apparatus according to any one of supplementary notes D1 to D3, further executing output information generation processing in which the at least one processor generates output information for assisting a medical worker in decision making with reference to the score aggregated by the aggregation processing.

### Supplementary Note D7

An information processing apparatus in which the at least one processor executes:
a first generation processing of generating a triple group including a positive example triple and a negative example triple with reference to a knowledge graph,
a second generation processing of generating a sentence from each of the triples included in the triple group, and
a first learning processing of acquiring sentence embeddings obtained by using a trained natural language model for each of the generated sentences and causing a score calculation model for calculating a score of each of the sentence embeddings to be trained with reference to each of the sentence embeddings.

### Supplementary Note D8

The information processing apparatus according to supplementary note D7, in which the at least one processor executes:
a second learning processing of causing an embedding model for performing knowledge graph embedding to be trained with reference to each of the triples included in the triple group, and
a third learning processing of causing a link prediction model for performing link prediction with reference to the knowledge graph embedding to be trained with reference to each of the triples included in the triple group.

### Supplementary Note D9

The information processing apparatus according to supplementary note D8, in which the at least one processor executes a fourth learning processing of causing an aggregation model for calculating an aggregated score from the score calculated by the score calculation model and the score calculated by the link prediction model to be trained.

### Supplementary Note E

The present disclosure includes the technologies described in the following supplementary notes. However, the present disclosure is not limited to the technologies described in the following supplementary notes, and various modifications can be made within the scope described in the claims.

### Supplementary Note E1

A non-transitory computer readable medium recorded with an information processing program for causing execution of:
an acquisition processing of acquiring a query,
a first generation processing of generating one or a plurality of triples with reference to the query,
a second generation processing of generating a sentence from each of the one or the plurality of triples,
a first calculation processing of acquiring sentence embeddings obtained by using a trained natural language model for each of the generated sentences and calculating a score of each of the sentence embeddings,
a second calculation processing of calculating a score of knowledge graph embedding of each of the one or the plurality of triples, and
an aggregation processing of aggregating the score calculated by the first calculation processing and the score calculated by the second calculation processing.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

What is claimed is:

1. An information processing apparatus comprising:
at least one memory storing instructions; and
at least one processor configured to execute the instructions to:
acquire a query;
perform a first generation of generating one or a plurality of triples with reference to the query;
perform a second generation of generating a sentence from each of the one or plurality of triples;
acquire sentence embeddings obtained by using a trained natural language model for each of the generated sentences and perform a first calculation of calculating a score of each of the sentence embeddings;
perform a second calculation of calculating a score of knowledge graph embedding of each of the one or plurality of triples; and
aggregate the score calculated in the first calculation and the score calculated in the second calculation.

2. The information processing apparatus according to claim 1, wherein the at least one processor is further configured to execute the instructions to, in the first calculation, calculate a score of the sentence embeddings by inputting each of the sentence embeddings to a score calculation model learned by machine learning.

3. The information processing apparatus according to claim 2, wherein the at least one processor is further configured to execute the instructions to:
in the second calculation,
calculate a knowledge graph embedding of each of the one or plurality of triples; and
calculate the score by executing link prediction related to each of the one or plurality of triples with reference to the calculated knowledge graph embedding.

4. The information processing apparatus according to claim 1, wherein the natural language model is a model learned by machine learning with reference to learning data in a medical field or a biochemical field.

5. The information processing apparatus according to claim 1, wherein the query includes information regarding at least any of a regimen, a chemical agent, a gene, and a disease.

6. The information processing apparatus according to claim 1, wherein the at least one processor is further configured to execute the instructions to generate output information for assisting a medical worker in decision making with reference to scores aggregated in the aggregation.

7. An information processing apparatus comprising:
at least one memory storing instructions; and
at least one processor configured to execute the instructions to:
perform a first generation of generating a triple group including a positive example triple and a negative example triple with reference to a knowledge graph;
perform a second generation of generating a sentence from each of the triples included in the triple group; and
acquire sentence embeddings obtained by using a trained natural language model for each of the generated sentences and perform a first learning of causing a score calculation model for calculating a score of each of the sentence embeddings to be trained with reference to each of the sentence embeddings.

8. The information processing apparatus according to claim 7, wherein the at least one processor is further configured to execute the instructions to:
perform a second learning of causing an embedding model for performing knowledge graph embedding to be trained with reference to each of the triples included in the triple group; and
perform a third learning of causing a link prediction model for performing link prediction with reference to the knowledge graph embedding to be trained with reference to each of the triples included in the triple group.

9. The information processing apparatus according to claim 8, wherein the at least one processor is further configured to execute the instructions to perform a fourth learning of causing an aggregation model for calculating an aggregated score from the score calculated by the score calculation model and the score calculated by the link prediction model to be trained.

10. An information processing method comprising:
acquiring a query;
performing a first generation of generating one or a plurality of triples with reference to the query;
performing a second generation of generating a sentence from each of the one or plurality of triples;
performing a first calculation of acquiring sentence embeddings obtained by using a trained natural language model for each of the generated sentences and calculating a score of each of the sentence embeddings;
performing a second calculation of calculating a score of knowledge graph embedding of each of the one or plurality of triples; and
aggregating a score of the sentence embeddings and a score of the knowledge graph embedding.

11. The information processing method according to claim 10, wherein the first calculation includes calculating a score of the sentence embeddings by inputting each of the sentence embeddings to a score calculation model learned by machine learning.

12. The information processing method according to claim 11,
wherein the second calculation includes:
calculating a knowledge graph embedding of each of the one or plurality of triples, and
calculating the score by executing link prediction related to each of the one or plurality of triples with reference to the calculated knowledge graph embedding.

13. The information processing method according to claim 10, wherein the natural language model is a model learned by machine learning with reference to learning data in a medical field or a biochemical field.

14. The information processing method according to claim 10, wherein the query includes information regarding at least any of a regimen, a chemical agent, a gene, and a disease.

15. The information processing method according to claim 10, further including output information generation that generates output information for assisting a medical worker in decision making with reference to the aggregated score.
